(19) United States
(12) Patent Application Publication
(10) Pub. No.: US 2005/0202433 A1
(43) Pub. Date: Sep. 15, 2005

(54) Novel high density arrays and methods for analyte analysis

(76) Inventor: Marinus Gerardus Johannus Van Beuningen, Oss (NL)

Correspondence Address: AMSTER, ROTHSTEIN & EBENSTEIN LLP, 90 PARK AVENUE, NEW YORK, NY, 10016, US

(21) Appl. No.: 10/515,485

(22) PCT Filed: Jun. 2, 2003
(86) PCT No.: PCT/EP03/05749

§ 371 Date: May 23, 2005

(30) Foreign Application Priority Data
Jun. 3, 2002 (EP) 02447108.8

(51) Int. Cl. C12Q 1/68; C12M 1/34

(52) U.S. Cl. 435/6; 435/287.2

(57) ABSTRACT

The present invention relates to methods for identifying analytes in a sample comprising the steps of: (a) incubating said analytes with a plurality of bipartite capture probes, said capture probes being immobilized in predefined regions on a solid substrate, and each capture probe consisting essentially of a first fragment which is at one end immobilized to said substrate and at the other end is complementary linked to a second fragment, wherein said second fragment comprises an extension fragment capable of identifying an analyte; (b) monitoring complex formation between sample analytes and extension fragments; (c) sequentially modifying complex formation conditions; allowing the release of captured analyte molecules from the substrate; and (d) detecting and identifying the released analytes. The present invention also relates to different uses of said methods as well as microarrays and kits for performing said methods.

### FIELD OF THE INVENTION

The present invention relates to the field of molecular biology and is particularly concerned with the technique of microarrays used for detection of molecules of interest in a sample, determination of composition of a complex mixture of molecules, and comparison of composition of two or more samples of molecules. The present invention relates to a method for optimizing microarray capacity of analyte analysis on an array of target molecules. The present invention is applicable to high-throughput genotyping of known and unknown polymorphisms and mutations.

### BACKGROUND TO THE INVENTION

During the past decade, the development of array-based analysis and identification technology has received great attention. This high throughput method, in which hundreds to thousands of molecules or probes immobilized on a solid substrate are hybridized to analyte molecules to gain, among others, kinetic, sequence, concentration and function information, has brought economical incentives to many applications.

DNA microarrays, consisting of high-density arrangements of oligonucleotides or complementary DNAs (cDNAs) can be used to interrogate complex mixtures of molecules in a parallel and quantitative manner.

The applications of the microarrays are driven by their increasing use in diagnostic testing and genomic research at academic institutions, biotechnology and pharmaceutical companies. In recent years, the main driver has been genomic analysis.

One application of the array technology is the genotyping of mutations and polymorphisms, also known as re-sequencing. With the availability of gene sequences from various eukaryotic and prokaryotic species and their genetic variations in terms of single nucleotide polymorphisms (SNP), polymorphisms, haplotypes or others, there is an increase in performing sequence variation analysis and coupling of these to, for example, large-scale drug population screenings towards the study, diagnosis, and treatment of genetic diseases. Ideally, all sequence variations would need to be analyzed for e.g. disease linkage. This requires high-density arrays.

Typically, 2-dimensional microarrays are generated on glass substrates. The microarrays are created by depositing molecules of interest on one surface of the glass substrate in pre-defined regions or spots, wherein a single spot can contain one or more molecule species.

The number of molecules on an array is limited by the amount of active surface area available. The development of 3-dimensional arrays have substantially increased the active surface area for arrays of molecules. Such type of arrays have been recently disclosed in e.g. U.S. 20020051995A1 or U.S. Pat. No. 6,383,742 which describe 3-D microarrays fabricated by stacking multiple 2-dimensional arrays. Other 3D microarrays have been manufactured by arraying beads or particles as mentioned in WO 02/38812.

The most important limitations of current technologies include high cost of manufacture and requirement of specialized and expensive instrumentation.

It is therefore an object of the present invention to provide a much improved 3D-microarray based methods for efficient, fast, and cost-effective analyte analysis.

It is a further object of the present invention to provide a microarray for performing said methods.

The present invention also aims at providing kits for performing said methods.

### SUMMARY OF THE INVENTION

The present invention relates to microarray analysis of analytes in a sample. The method according to the present specification employs a 3D microarray comprising high active surface content. Compared to known 2D substrates, the substrate as employed in the present specification has at least a 500-fold enlarged active surface area. In order to make efficient use of said enlarged area, predefined regions of the substrate are spotted with combinations of distinct capture probes. Based on the increased surface area, the amount of material spotted per probe is the same as compared to a flat surface array, assuming equal binding conditions. The unique composition of each distinct capture probe in a predefined region allows for the sequential detection of bound analytes.

The present invention provides a method for identifying analytes in a sample comprising the steps of:
(a) incubating said analytes with a plurality of bipartite capture probes, said capture probes being immobilized in predefined regions on a solid substrate, and each capture probe consisting essentially of a first fragment which is at one end immobilized to said substrate and at the other end is complementary linked to a second fragment, wherein said second fragment comprises an extension fragment capable of identifying an analyte;
(b) monitoring complex formation between sample analytes and extension fragments;
(c) sequentially modifying complex formation conditions; allowing the release of captured analyte molecules from the substrate; and
(d) detecting and identifying the released analytes.

An advantage of the present invention is the highly efficient use of the available active surface in a porous substrate, allowing a combination of up to 100 distinct probes, each, e.g., representing a genetic variant, in a single spot and the analysis of up to 300.000 spots per cm².

Additional features and advantages of the invention will be set forth in the detailed description which follows, and in part will be apparent from the description, or may be learned by practice of the invention. The objectives and other advantages of the invention will be realized and attained by the process particularly pointed out in the written description and appended claims.

### DETAILED DESCRIPTION OF THE INVENTION

The present invention relates to methods and corresponding high capacity arrays for analysis of analytes in a sample. The invention described herein addresses the unmet needs in the art for accurate detection and determination of concentration of a variety of compounds or molecules in solution, using an array-based assay.

In the present specification and the appended claims, the singular forms “a”, “an”, and “the” include plural references unless the context clearly dictates otherwise. Unless defined otherwise, all technical and scientific terms used herein have the same meaning as commonly understood to one of ordinary skill in the art.

The terms “analyte” and “analyte molecule” are used interchangeably throughout the present invention. The term “analyte in a sample” refers to a molecule in a sample, i.e. a molecule to be analysed.

An analyte as used in the present specification refers to any molecule which may associate or bind to a target-molecule immobilized onto a porous substrate for the purpose of performing micro-array analysis. The term analyte as used in the present specification refers both to separate molecules and to portions of molecules such as e.g. an epitope of a protein.

Examples of analytes which may be employed in the present invention include, but are not limited to, antibodies including monoclonal antibodies polyclonal antibodies, purified antibodies, synthetic antibodies, antisera reactive with specific antigenic determinants (such as viruses, cells or other materials), proteins, peptides, polypeptides, enzyme binding sites, cell membrane receptors, lipids, proteolipids, drugs, polynucleotides, oligonucleotides, sugars, polysaccharides, cells, cellular membranes and organelles, nucleic acids including deoxyribonucleic acids (DNA), ribonucleic acids (RNA), and peptide nucleic acids (PNA) or any combination thereof; cofactors, lectins, metabolites, enzyme substrates, metal ions and metal chelates.

Virtually any sample may be analyzed using the method according to the present specification. However, usually, the sample is a biological or a biochemical sample. The term “biological sample,” as used herein, refers to a sample obtained from an organism or from components (e.g., cells) of an organism. The sample may be of any biological tissue or fluid. Frequently the sample will be a “clinical sample” which is a sample derived from a patient. Such samples include, but are not limited to, sputum, cerebrospinal fluid, blood, blood fractions such as serum including fetal serum (e.g., SFC) and plasma, blood cells (e.g., white cells), tissue or fine needle biopsy samples, urine, peritoneal fluid, and pleural fluid, or cells there from. Biological samples may also include sections of tissues such as frozen sections taken for histological purposes.

Examples of biochemical samples include, without limitation, cell line cultures, purified functional protein solutions, polypeptide solutions, nucleic acid solutions including oligonucleotide solutions, and others.

Samples may be analyzed directly or they may be subject to some preparation prior to use in the assays of this invention. Non-limiting examples of said preparation include suspension/dilution of the sample in water or an appropriate buffer or removal of cellular debris, e.g. by centrifugation, or selection of particular fractions of the sample before analysis. Nucleic acid samples, for example, are typically isolated prior to assay and, in some embodiments, subjected to procedures, such as reverse transcription and/or amplification (e.g., polymerase chain reaction, PCR) to increase the concentration of all sample nucleic acids (e.g., using random primers) or of specific types of nucleic acids (e.g., using polynucleotide-thymidylate to amplify messenger RNA or gene-specific primers to amplify specific gene sequences). The amplification method set out in WO 99/43850 may also be used in the present invention.

The terms “probe” and “capture probe” are used interchangeably throughout the present invention and refer to the immobilized molecules that are capable of capturing on or more analyte molecules by specifically binding thereto. An “Immobilized molecule” means a molecule that can be immobilized on a substrate by any means conventional in the art.

The present invention is based on the unique composition of each bipartite capture probe within a predefined region.

Accordingly, in one embodiment of the present invention, a method is provided wherein each predefined region on the substrate as used in said method comprises a plurality of distinct capture probes. The number of distinct capture probes within a single predefined region may be comprised between 2 and 100, or more.

The terms “spot” and “predefined region” are used interchangeably throughout the present invention and relate to individually, spatially addressed positions on the substrate to form an array.

For a given substrate size, the upper limit of number of spots on a substrate is determined by the ability to create and detect spots in the array. The preferred number of spots on an array generally depends on the particular use to which the array is to be put. For example, sequencing by hybridization will generally require large arrays, while mutation detection may require only a small array. In general, arrays contain from 2 to 106 spots and more, or from about 100 to about 105 spots, or from about 400 to about 110 spots, or between about 500 and about 2000 spots.

A probe set as used in a single predefined region consists of specific hybridized molecules comprising characteristic interacting regions. For each bipartite probe, at least 3 specific interacting regions may be distinguished. The term “specific interacting region” as used in the present specification refers to molecules or parts of molecules with an inherent or artificially created property to recognize and selectively bind another molecule. Non-limiting examples of such recognition and specific bonds include hybridization of complementary oligonucleotides, polynucleotides, or nucleic acids, or synthetic molecules chemically synthesized to bind to other molecules.

The bipartite probes of the present invention are composed of a first and a second fragment. A first specific interaction region is found within the first fragment which is immobilized to the substrate by its 5′ end. Said 5′ end may be a linker molecule.

Accordingly, in one embodiment of the present invention, a method is provided, wherein said first fragment of a bipartite probe is immobilized to the substrate by a linker molecule.

Suitable linkers include, by way of example and not limitation, polypeptides such as polyproline or polyalanine, saturated or unsaturated bifunctional hydrocarbons such as 1-amino-hexanoic acid, polymers such as polyethylene glycol, etc., 1,4-Dimethoxytrityl-polyethylene glycol phosphoramidites useful for forming phosphodiester linkages with hydroxyl groups and are described, for example in Zhang et al., 1991, Nucl. 20 Acids Res. 19:3929-3933 and Durand et al., 1990, Nucl. Acids Res. 18:6353-6359. Other useful linkers are commercially available.

The expression “immobilized on a substrate” as used in the present specification refers to the attachment or adherence of one or more target molecules to the surface of a porous substrate including attachment or adherence to the inner surface of said substrate.

Molecules or compounds may be immobilized either covalently (e.g., utilizing single reactive thiol groups of cysteine residues,) or non-covalently but specifically (e.g., via immobilized antibodies, the biotin/streptavidin system, and the like), by any method known in the art. Further examples of the various methods that are available to attach target molecules to porous substrates include but are not limited to biotin-ligand non-covalently complexed with streptavidin, S—H-ligand covalently linked via an alkylating reagent such as an iodoacetamide or maleimide, amine-ligand covalently linked via an activated carboxylate group (e.g., EDAC coupled, etc.), phenylboronic acid (PBA) ligand complexed with salicylhydroxamic acid (SHA), and acrylic linkages allowing polymerization with free acrylic acid monomers to form polyacrylamide or reaction with SH or silane surfaces. More specifically, immobilization of proteins may be accomplished through attachment agents selected from the group comprising cyanogen bromide, succinimides, aldehydes, tosyl chloride, avidin-biotin, photo-crosslinkable agents including hetero bifunctional cross-linking agents such as N-[y-maleimidobutyryloxylsuccinimide ester (GMBS), epoxides, and maleimides. Antibodies may be attached to a porous substrate by chemically cross-linking a free amino group on the antibody to reactive side groups present within the support. For example, antibodies may be chemically cross-linked to a substrate that contains free amino, carboxyl, or sulfur groups using glutaraldehyde, carbo-di-imides, or hetero bi-functional agents such as GIVMS as cross-linkers.

In one embodiment of the present invention, capture probes are immobilized to the solid substrate by means of covalent bonding.

Covalent linkage to a substrate is well known in the art. Covalent binding of an organic compound to a metal oxide is well known in the art, for example using the method described by Chu. C. W., et al (J. Adhesion Sci. Technol., 7, pp. 417-433; 1993) and Fadda, M. B. et al. (Biotechnology and Applied Biochemistry, 16, pp. 221-227, 1992).

In order to introduce distinction between capture probes within a single predefined region, the 5′ ends or linker molecules of the first fragments may comprise a breakable region. A variety of breakable regions among said 5′ or linker ends allow sequential release of the immobilized molecules from the substrate upon subjection of the substrate with corresponding appropriate release treatments. Said treatments may include, by way of example and not limitation, chemical treatments such as disulphide bridge disruption, acid hydrolysis, and light radiation treatments to act on light-activatable groups.

Accordingly, in one embodiment of the present invention, a linker molecule is chosen from the group of stable or labile linker molecules.

In a further embodiment, said linker molecule is a labile linker.

In yet a further embodiment, said linker molecule is chosen from the group comprising physically labile and chemically labile linkers.

In yet a further embodiment, said labile linker is chosen from the group comprising photo-labile, acid-labile, base-labile, enzyme-labile, and oxidation-labile linkers.

A second specific interaction region allows a second fragment of a bipartite probe to hybridize to a first fragment through complementary nucleic acid sequences of both first and second fragments. Therefore, distinction between individual capture probes within a predefined region may, alternatively, be introduced by way of sequence variation within the complementary hybridizing regions of first and second fragments of said individual probes. Such sequence variation lead to different melting temperatures. These regions are therefore referred to as temperature tag sequences of first and second fragments.

For simplicity, temperature tag sequence as used in the present specification refers to the single stranded sequences as present within the first and second fragments of the bipartite probes but also refers to the double strand complementary overlap region between first and second fragments.

Accordingly, in one embodiment of the present invention, said first fragment is complementary linked to said second fragment by a temperature tag sequence.

Typically, within the context of the present invention, said temperature tag sequences comprise from 10 up to 40 or more nucleotides. The introduced sequence variation results in different melting temperatures and hence, subjection of the substrate to temperature variation will affect the different first fragment/second fragment hybridizations within the different temperature tag sequences.

A distinction between individual capture probes within a predefined region may also be introduced by way of providing a restriction enzyme recognition region within the temperature tag sequence.

The probe characteristics defined by linker molecules and/or temperature tag sequences which, in essence, make up the first fragments, allow distinct capture probes within a predefined region to specifically release the bound analyte upon releasing conditions defined by said linker molecules and/or temperature tag sequences.

Therefore, in one embodiment of the present invention, a method is provided, wherein each distinct capture probe immobilized in a predefined region differs in analyte releasing condition.

In a further embodiment, said analyte releasing condition is defined by said temperature tag or said linker molecule or a combination thereof.

Accordingly, in another embodiment of the present invention, the sequential release of captured analyte molecules from the substrate is by a modifying condition chosen from the group comprising temperature variation, base treatment, oxidative treatment, enzymatic treatment, and photolysis, including any combination thereof.

In order to analyse anayltes in a sample, the second fragment of the bipartite probe comprises an extension fragment capable of identifying, by specific binding, an analyte. This third interacting region of the bipartite probe may be a nucleic acid.

Accordingly, in one embodiment of the present invention, a method is provided, wherein said extension fragment is a nucleic acid sequence.

The extension nucleic acid fragment is sufficiently long to have a high enough Tₘ with a bound analyte such that said nucleic acid/analyte interaction cannot be released upon subjection of the substrate to a target releasing condition as described above; i.e. a target releasing condition releases either a second fragment/analyte complex (e.g. upon temperature variation) or a first fragment/second fragment/analyte complex (e.g. upon breakage of the linker molecule). Particularly suitable nucleic acid extension fragments may be 30 to 80 nucleotides in length.

Long extension fragments, as such, and as provided in one embodiment of the present invention, provide for extension fragment/analyte nucleic acid hybrids with high Tₘ values.

In a further embodiment, said high Tₘ of an extension fragment/analyte nucleic acid complex as obtained by a method according to the present invention is substantially higher than the Tₘ as defined by the temperature tag sequences.

Accordingly, in one embodiment of the present invention, temperature variation, as modifying condition as described above, is by means of detecting at subsequent higher Tₘ values, said Tₘ values corresponding to the Tₘ values as defined by the temperature tag sequences of the capture probes, and whereby said temperature variation does not affect the extension fragment/analyte interaction.

In a further embodiment of the present invention, said nucleic acid sequence is an oligonucleotide.

By “oligonucleotide” or “oligonucleotide sequence” is meant a nucleic acid of a length of about 6 to about 150 or more bases. Oligonucleotides are generally, but not necessarily, synthesized in vitro. A segment of nucleic acid that is 6 to 150 bases and that is a subsequence of a larger sequence may also be referred to as an oligonucleotide sequence.

The term oligonucleotide refers to a molecule comprised of one or more deoxyribonucleotides, such as primers, probes, and nucleic acid fragments.

In a further embodiment of the present invention, nucleic acid extension fragments comprise a stem-loop sequence.

In yet a further embodiment, said stem-loop sequence is a molecular beacon. Molecular beacons consist essentially of a fluorescent donor, an analyte binding or identifying sequence, and a quencher.

The term “fluorescent donor” refers to the radical of a fluorogenic compound which can absorb energy and is capable of transferring the energy to another fluorogenic molecule or part of a compound. Suitable donor fluorogenic molecules include, but are not limited to, coumarins and related dyes, xanthene dyes such as fluoresceins, rhodols, and rhodamines, resorufins, cyanine dyes, bimanes, acridines, isoindoles, dansyl dyes, aminophthalic hydrazides such as luminol and isoluminol derivatives, aminophthalimides, aminonaphthalimides, aminobenzofurans, aminoquinolines, dicyanohydroquinones, and europium and terbium complexes and related compounds.

The term “quencher” refers to a chromophoric molecule or part of a compound which is capable of reducing the emission from a fluorescent donor when attached to the donor. Quenching may occur by any of several mechanisms including fluorescence resonance energy transfer, photo-induced electron transfer, paramagnetic enhancement of intersystem crossing, Dexter exchange coupling, and excitation coupling such as the formation of dark complexes. A quencher may operate via fluorescence resonance energy transfer. Many quenchers can re-emit the transferred energy as fluorescence. Examples include coumarins and related fluorophores, xanthenes such as fluoresceins, rhodols, and rhodamines, resorufins, cyanines, difluoroboradiazaindacenes, and phthalocyanines. Other chemical classes of quenchers generally do not re-emit the transferred energy. Examples include indigos, benzoquinones, anthraquinones, azo compounds, nitro compounds, indoanilines, di- and triphenylmethanes.

The term “dye” refers to a molecule or part of a compound that absorbs specific frequencies of light, including but not limited to ultraviolet light. The terms “dye” and “chromophore” are synonymous.

The term “fluorophore” refers to a chromophore that fluoresces.

The use of stem-loop or molecular beacon sequences enables the use of multiple fluorophores and multiple analysis per spot. This allows the first scanning of, for example, four different fluorophore channels for all probes and analytes bound in a given spot at low temperature. Subsequently, a temperature variation may be installed, e.g. an increase in temperature, and again all fluorescent channels at said increased temperature are scanned.

Non-limiting examples of suitable fluorophores include include, by way of example and not limitation, fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy X-rhodamine (ROX), 6-carboxy-2′,4′,7′,4,7-hexachlorofluorescein (HEX), 5-carboxyfluorescein (5-FAM), N,N,N′,N′-tetramethyl-6-carboxyrhodamine (TAMRA), cyanine dyes (e.g. Cy5, Cy3), BODIPY dyes (e.g. BODIPY 630/650, Alexa542, etc), green fluorescent protein (GFP), blue fluorescent protein (BFP), yellow fluorescent protein (YFP), red fluorescent protein (RFP), and the like, (see, e.g., Molecular Probes, Eugene, Oreg., USA).

Accordingly, in one embodiment of the present invention, a method is provided wherein different signals may be detected at a single release condition.

In a further embodiment, a method is provided, wherein different signals may be detected within a single predefined region at a single release condition.

In another embodiment of the present invention, a method is provided wherein the analyte molecules comprise a label, said label capable of generating an identifiable signal.

Fluorescent labels are particularly suitable because they provide very strong signals with low background. Fluorescent labels are also optically detectable at high resolution and sensitivity through a quick scanning procedure. Fluorescent labels offer the additional advantage that irradiation of a fluorescent label with light can produce a plurality of emissions. Thus, a single label can provide for a plurality of measurable events.

Accordingly, in a particular embodiment, said label is a fluorophore.

Detectable signal may equally be provided by chemiluminescent and bioluminescent labels. Chemiluminescent sources include compounds which becomes electronically excited by a chemical reaction and can then emit light which serves as the detectable signal or donates energy to a fluorescent acceptor. Alternatively, luciferins can be used in conjunction with luciferase or lucigenins to provide bioluminescence.

Temperature variation may be continuous or stepwise. A suitable example of a stepwise temperature increase in the method according to the present invention, is a Tₘ increase by no more than 15° C. at each subsequent increment. A more suitable example of a stepwise temperature increase in the method according to the present invention, is a Tₘ increase by no more than 10° C. A particular suitable example of a stepwise temperature increase in the method according to the present invention, is a Tₘ increase by no more than 5° C.

The term ‘solid substrate’ refers to any solid substrate conventional in the art that supports an array and on which molecules are allowed to interact and their reaction detected without degradation of or reaction with its surface. The surface of the substrate may be a bead or particle such as microspheres or nanobeads, or planar glass, a flexible, semi-rigid or rigid membrane, a plastic, metal, or mineral (e.g., quartz or mica) surface, to which a molecule may be adhered. The solid substrate may be planar or have simple or complex shape. The surface to which the target molecules or probes are adhered can be the external surface or the internal surface of the solid substrate. Particularly, where the substrate is porous by nature or by manufacturing practices, the molecules are likely to be attached to an internal surface.

The terms “adhered to” or “attached to” a solid substrate denotes that the first binding molecules are directly or indirectly fixed to the solid substrate.

Generally, the substrate according to the present invention may be composed of any porous material which will permit immobilization of a target molecule and which will not melt or otherwise substantially degrade under the reaction conditions used. The surface to which the molecule is adhered may be an external surface or an internal surface of the porous substrate. In particular, in the present invention, the internal surface of a porous substrate may be maximally occupied by sets of distinct molecules or capture probes.

The term “active surface” refers to the substrate surface which may have immobilized target molecules thereon. Said active surface may be the external or the internal surface.

A porous substrate, as used in the present invention, may be manufactured out of, for example, a metal, a ceramic metal oxide or an organic polymer. In view of strength and rigidity, a metal or a ceramic metal oxide may be used. Above all, in view of heat resistance and chemicals resistance, a metal oxide may be used. In addition, metal oxides provide a substrate having both a high channel density and a high porosity, allowing high density arrays comprising different first binding substances per unit of the surface for sample application. In addition, metal oxides are highly transparent for visible light. Metal oxides are relatively cheap substrates that do not require the use of any typical microfabrication technology and, that offers an improved control over the liquid distribution over the surface of the support, such as an electrochemically manufactured metal oxide membrane. Metal oxide membranes having through-going, oriented channels can be manufactured through electrochemical etching of a metal sheet.

Accordingly, in one embodiment of the present invention, a method is provided as described herein, wherein said solid substrate is a metallo-oxide substrate.

The kind of metal oxide is not especially limited, but can be preferably used. As a metal, for example, a porous substrate of stainless steel (sintered metal) can be used. For applications not requiring heat resistance, a porous substrate of an organic polymer can also be used if it is rigid.

Metal oxides considered are, among others, oxides of zirconium, silica, mullite, cordierite, titanium, zeolite or zeolite analog, tantalum, and aluminum, as well as alloys of two or more metal oxides and doped metal oxides and alloys containing metal oxides.

In one embodiment, a method as described herein is provided, wherein said solid substrate is an aluminum-oxide substrate.

The metal oxide membranes are transparent, especially if wet, which allows for assays using various optical techniques. Such membranes have oriented through-going channels with well-controlled diameter and useful chemical surface properties. WO 99/02266 which discloses the Anopore™ porous substrate is exemplary in this respect, and is specifically incorporated in the present invention.

The porous nature of the substrate facilitates the pressurized movement of fluid, e.g. the sample solution, through its structure. In contrast to two-dimensional substrates, the flow-through nature of a 3-dimensional substrate or microarray, as employed in the methods as described herein, gives significantly reduced hybridization times and increased signal and signal-to-noise ratios. Further, a positive or negative pressure may be applied to the arrays in order to pump the sample solution dynamically up and down through the substrate pores.

In a further embodiment, a method as described herein is provided wherein said solid substrate is a flow-through substrate.

Particularly suitable applications for the methods as described herein, include genotyping. Thereto, and in a specific embodiment of the present invention, nucleic acid extension fragments of the second fragments of the bipartite probes comprise a nucleic acid mutation site.

In a further embodiment, said nucleic acid mutation site is chosen from the group comprising deletions and insertions, including frame-shift mutations; and base pair substitutions, including single nucleotide mutations.

In a particular embodiment, said nucleic acid mutation site is a single nucleotide polymorphism.

It is a further object of the present invention to provide microarrays for performing a method as described herein, comprising a solid substrate, said solid substrate having immobilized thereon a set of distinct bipartite capture probes, said set of distinct capture probes being sub-divided in sub-sets of distinct capture probes, wherein each said subset of distinct capture probes is immobilized within a predefined region on said solid substrate, and wherein each distinct capture probe within a single predefined region comprises a distinct first fragment which is at one end immobilized to the substrate and to the other end complementary linked to a second fragment, wherein said second fragment comprises an extension fragment capable of identifying an analyte.

In one embodiment, such a microarray is provided wherein capture probes are immobilized to the solid substrate by means of covalent bonding.

In a further embodiment, a microarray as described herein is provided wherein the solid substrate is an aluminum oxide substrate.

In a yet a further embodiment, a microarray as described herein is provided wherein said solid substrate is a flow-through substrate.

In a yet a further embodiment, the use of a microarray as described herein is provided for the manufacture of a nucleic add analysis kit.

It is a further object of the present invention to provide a kit for performing a method as described herein, comprising:
(a) a microarray as provided by the present invention;
(b) a set of bipartite capture probes, said capture probes characterized by a first fragment consisting essentially of a linker molecule and a temperature tag sequence, said temperature tag sequence hybridizing with a second fragment, said second fragment comprising an extension fragment capable of identifying an analyte.

In one embodiment, a kit is provided, wherein said extension fragment comprises a nucleic acid mutation site selected from the group comprising deletions and insertions, including frame-shift mutations; and base-pair substitutions, including single nucleotide mutations.

It is a further object of the present invention to provide for the use of a method as described herein, for detecting nucleotide variations in a nucleic acid sample, said variations selected from the group comprising deletions and insertions, including frame-shift mutations; and base-pair substitutions, including single nucleotide mutations or polymorphisms.

In one embodiment, the present invention provides for the use of a method as described herein, for kinetic monitoring of a multitude of Tₘ dependent nucleic acid hybridization events.

The following figures and examples serve to illustrate the present invention but are in no way construed to be limiting the present invention.

### SHORT DESCRIPTION OF THE FIGURES

FIG. 1 illustrates a set of five bipartite capture probes 1, 2, 3, 4, and 5 which is present in a predefined region on a microarray according to the present invention. Each bipartite probe consists essentially of a first fragment which is immobilized to the substrate by a linker molecule (A). Said first fragment is, at its 3′ end, complementary linked to a second fragment by a temperature tag sequence (B). Said second fragment comprises an extension fragment (C) which is capable of identifying an analyte (D) in a sample. Said extension fragment may comprise a stem-loop or molecular beacon sequence (E) which consist essentially of a fluorescent donor (Fl), an analyte binding or identifying sequence, and a quencher (O). The temperature tag sequence (B) may have a recognition site for a restriction enzyme (RE).

FIG. 2 illustrates the hybridiation signals which are obtained when a sequential temperature variation is applied to the array of captured analyte/probe complexes. The signals obtained are the sums of individual signals generated by analytes which are captured by probes with different temperature target release conditions. For example, at low temperatures (e.g. 40° C.) the overall signal is the sum of the signals generated from the analytes which are bound to capture probes 1, 2, 3, and 4 as described in FIG. 1. At sequentially higher temperatures, said signal will be modified according to the sequential release of labeled extension fragment/analyte complexes from the substrate.

### EXAMPLES

### Example 1

### Detection of Nucleic Acid Sequence Variations in a Sample

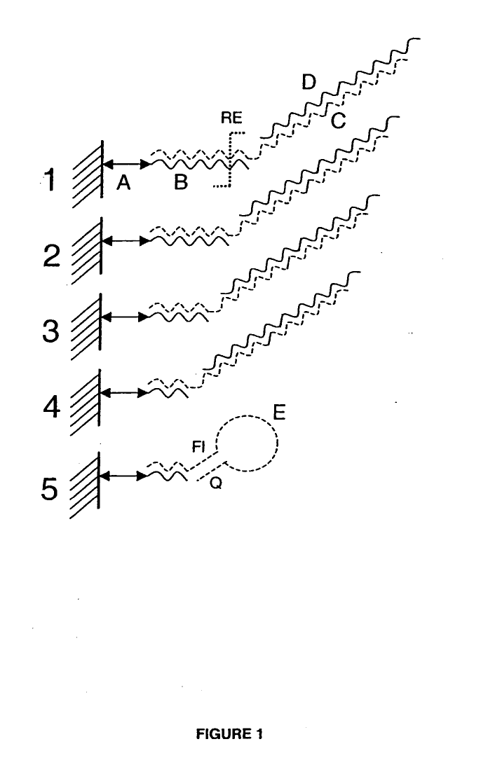

An array of capture probe sets is used to detect a number of 1000-10000 SNP's or other known sequences using a limited number of features on a metal oxide substrate. The capture probe set sequences are constructed and blasted to GenBank® Database sequences. Each first fragment of a bipartite probe consists of a 5′-prime linking moiety (“A” in FIG. 1) thiol or amine or carboxyl or a photo-reactive linkage. Each first fragment comprises a temperature tag sequence with length of 10-30 nucleotides (“B” in see FIG. 1) and has a binding region (“RE” in FIG. 1) for a restriction enzyme. A set of first fragments is covalently coupled to the substrate as well-know in the art. A number of distinct first fragments is mixed together (1+2+3+4, see FIG. 1) to form a set of distinct first fragments which is covalently attached to a predefined region or spot on the substrate. Each of these first fragments within a set has a different release region (e.g. chemical linkage of linker molecule A, sequence length of temperature tag B). After manufacturing of the arrays, a mixture of complementary second strand molecules (“C” in FIG. 1) is hybridised to the first fragment sets at a concentration of 0.1-10 nM in 5×SSPE at 30° C. The complementary second strand sequences consist essentially of a 5′-prime sequence complementary for the temperature tag sequences of the first strands and a 3′-prime extension fragment of 30-80 nucleotides which is complementary to sample nucleic acid sequences. The extension fragment may comprise a 5′-prime folded DNA sequence of which the 5′-prime end is hybridised with the end of the 3′ end of the extension fragment (capture probe 5 in FIG. 1). This enables the use of fluorescent dyes, which are quenched when present in their native folded state but give a strong fluorescent stain upon hybridisation to an analyte sequence.

After these steps the array is ready for hybridisation with the sample.

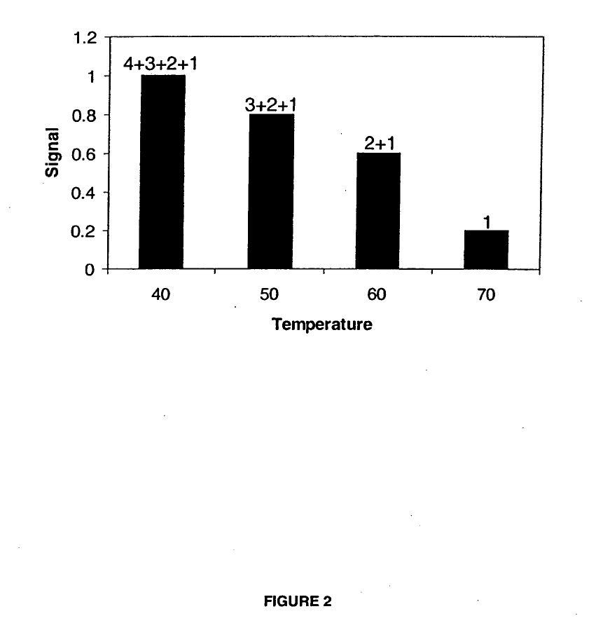

In the present example, the sample is a multiplex PCR sample, therein nucleic acids which are fluorescent primed or fluorescent labelled by incorporation of labelled nucleotides. The sample is purified using a spin column (Chroma Spin+TE30 columns and Microconr YM-30 columns). The sample, 20 μl, (0.1-100 nM) is hybridised at 40° C. for 15 minutes in 5×SSPE on the porous substrate with continuous pumping the sample twice up and down per minute through the substrate pores in the predefined regions. A CCD image is taken and analysed for spot intensity. The signal for a number of sample sequences on a capture probe set is shown in FIG. 2. The temperature is increased to 50° C. while continuously pumping of the sample. This temperature will first melt the sequence off the temperature tag of capture probe ‘4’ as shown in FIG. 1. A CCD image is taken and analysed for spot intensity. The difference between the signal taken at 40° C. and 50° C. is the signal specific for one of the sample sequences. The temperature is further increased to 60° C. and 70° C. and images are taken. The signal change is shown in FIG. 2.

A similar sequence of steps as done on the temperature is done with the use of sequential addition of restriction enzymes. Further, similar sequence of steps as done on the temperature is done by addition of chemical compounds, which selectively remove the coupling of first fragments. Furthermore another layer of analyte sequences is removed by the use of photolabile groups. The substrate is then illuminated with a UV light source to break the bond between a first fragment and the substrate.

The combination of temperature variation, chemical treatment steps, use of restriction enzymes and light degradation enables analysis of up to 100 different sample sequences in a given spot on the array.

1. A method for identifying analytes in a sample comprising the steps of:
(a) incubating said analytes with a plurality of bipartite capture probes, said capture probes being immobilized in predefined regions on a solid substrate, and each capture probe consisting essentially of a first fragment which is at one end immobilized to said substrate and at the other end is complementary linked to a second fragment, wherein said second fragment comprises an extension fragment capable of identifying an analyte;
(b) monitoring complex formation between sample analytes and extension fragments;
(c) sequentially modifying complex formation conditions; allowing the release of captured analyte molecules from the substrate; and
(d) detecting and identifying the released analytes.

2. The method according to claim 1, wherein said first fragment is complementary linked to said second fragment by a temperature tag sequence.

3. The method according to claim 1, wherein said first fragment is immobilized to said substrate by a linker molecule.

4. The method according to claim 1, wherein each predefined region comprises a plurality of distinct capture probes.

5. The method according to claim 4, wherein each distinct capture probe immobilized in said predefined region differs in analyte releasing condition.

6. The method according to claim 5, wherein said analyte releasing condition is defined by said temperature tag or said linker molecule or a combination thereof.

7. The method according to claim 3, wherein said linker molecule is a stable or a and labile linker molecule molecules.

8. The method according to claim 7, wherein said linker molecule is a labile linker.

9. The method according to claim 7, wherein said labile linker is a physically labile or a chemically labile linker.

10. The method according to claim 7, wherein said labile linker is a photo labile, an acid labile, a base labile, an enzyme labile, or an oxidation labile linker.

11. The method according to claim 1, wherein said sequentially releasing as defined in step (c) is by a modifying condition chosen from temperature variation, base treatment, acid treatment, oxidative treatment, enzymatic treatment, photolysis and any sequential combination thereof.

12. The method according to claim 11, wherein said temperature variation is by means of detecting at subsequent higher Tₘ values, said Tₘ values corresponding to the Tₘ values as defined by the temperature tag sequences of the capture probes, and whereby said temperature variation does not affect the extension fragment/analyte interaction.

13. The method according to claim 12, wherein the Tₘ is changed by no more than 15° C. at each subsequent increment.

14. The method according to claim 12, wherein the Tₘ is changed by no more than 10° C. at each subsequent increment.

15. The method according to claim 12, wherein the Tₘ is changed by no more than 5° C. at each subsequent increment.

16. The method according to claim 1, wherein said extension fragment as defined in step (b) is a nucleic acid sequence.

17. The method according to claim 16, wherein said nucleic acid sequence is an oligonucleotide.

18. The method according to claim 16, wherein said nucleic acid comprises a stem-loop sequence.

19. The method according to claim 18, wherein said stem-loop sequence is a molecular beacon.

20. The method according to claim 16, wherein said extension fragment/analyte nucleic acid has a high Tₘ.

21. The method according to claim 20, wherein said high Tₘ is substantially higher than the Tₘ defined by the temperature tag sequence as defined in claim 2.

22. The method according to claim 1, wherein said analyte comprises a label, said label capable of generating an identifiable signal.

23. The method according to claim 22, wherein said label is a fluorophore.

24. The method according to claim 1, wherein said extension fragment as defined in step (b) comprises a nucleic acid mutation site.

25. The A method according to claim 24, wherein said nucleic acid mutation site is a deletion, an insertion, a frame shift mutation, a base pair substitution or a single nucleotide mutation.

26. The method according to claim 25, wherein said nucleic acid mutation site is a single nucleotide polymorphism.

27. The method according to claim 1, wherein said immobilization of said capture probes to said solid substrate is by means of covalent bonding.

28. The method according to claim 1, wherein different signals may be detected at a single release condition.

29. The method according to claim 1, wherein different signals may be detected within a single predefined region at a single release condition.

30. The method according to claim 1, wherein said solid substrate is a metallo-oxide substrate.

31. The method according to claim 30, wherein said solid substrate is an aluminum-oxide substrate.

32. The method according to claim 1, wherein said solid substrate is a flow-through substrate.

33. Use of a method according to claim 1, for detecting nucleotide variations in a nucleic acid sample, said variations comprising deletions, insertions, frame-shift mutations, base-pair substitutions, single nucleotide mutations or polymorphisms.

34. Use of a method according to claim 1, for kinetic monitoring of a multitude of Tₘ dependent nucleic acid hybridization events.

35. A microarray comprising a solid substrate, said solid substrate having immobilized thereon a set of distinct bipartite capture probes, said set of distinct capture probes being sub-divided in sub-sets of distinct capture probes, wherein each said subset of distinct capture probes is immobilized within a predefined region on said solid substrate, and wherein each distinct capture probe within a single predefined region comprises a distinct first fragment which is at one end immobilized to the substrate and to the other end complementary linked to a second fragment, wherein said second fragment comprises an extension fragment capable of identifying an analyte.

36. The microarray according to claim 35, wherein said capture probes are immobilized to said solid substrate by means of covalent bonding.

37. The microarray according to claim 35 or 36, wherein said solid substrate is an aluminum oxide substrate.

38. The microarray according to claim 35, wherein said solid substrate is a flow-through substrate.

39. The use of a microarray according to claim 35, for the manufacture of a nucleic acid analysis kit.

40. A kit, comprising:
(e) a microarray according to claim 35;
(f) a set of bipartite capture probes, said capture probes characterized by a first fragment consisting essentially of a linker molecule and a temperature tag sequence, said temperature tag sequence hybridizing with a second fragment, said second fragment comprising an extension fragment capable of identifying an analyte.

41. The kit according to claim 40, wherein said extension fragment comprises a nucleic acid mutation site selected from deletions, insertions, frame-shift mutations and base-pair substitutions, and single nucleotide mutations.
